(19) United States
(12) Patent Application Publication
(10) Pub. No.: US 2021/0219970 A1
(43) Pub. Date: Jul. 22, 2021

(54) JOINT DISTRACTER

(71) Applicant: RAYCONT LTD., Yad Nathan (IL)

(72) Inventor: Gad SHMUELI, Yad Nathan (IL)

(21) Appl. No.: 17/225,546

(22) Filed: Apr. 8, 2021

Related U.S. Application Data
(63) Continuation of application No. PCT/IL2019/051114, filed on Oct. 10, 2019.
(60) Provisional application No. 62/743,638, filed on Oct. 10, 2018.

(51) Int. Cl. A61B 17/02

(52) U.S. Cl. CPC A61B 17/025; A61B 2017/0275; A61B 2562/0247; A61B 2562/0257; A61B 2562/0261

(57) ABSTRACT

According to an aspect of the invention there is provided a joint distractor, including: a stationary portion configured to fix a first body portion on a first side of a body joint, and an extendable portion configured to be urged against a second body portion on a second side of the body joint, and wherein extension of the extendable portion urges the extendable portion onto the second body portion away from the first body portion thereby forming a gap between articulating surfaces of the first and second body portions within the joint.

### CROSS REFERENCE TO RELATED APPLICATIONS

This application is a continuation of International Application No. PCT/IL2019/051114 to Shmueli (published as WO 2020-075175) filed Oct. 10, 2019, which claims the benefit of priority under 35 USC § 119(e) of U.S. Provisional Patent Application No. 62/743,638 filed Oct. 10, 2018 entitled “JOINT EXTENDER”. The contents of the above-referenced U.S. Provisional Patent Application are all incorporated herein by reference in their entirety.

### FIELD OF THE INVENTION

The invention relates generally to the field of joint distractors and, more particularly, but not exclusively, to self-contained portable joint distractors.

### BACKGROUND

The AcetabuloFemoral Joint (AFJ) is the articulation of the acetabulum with the femur, in which the femoral head pivots inside the acetabulum socket. Joint inflammation and trauma can induce articular cartilage damage and can lead to deterioration of the AFJ. Damaged or worn surfaces of the articular cartilage covering the femoral head or the acetabulum commonly lead to loss of the AFJ function, creating a need for orthopedic operations in which the articular cartilage surfaces are reconstructed, commonly named Resurfacing, or replaced, commonly named Total Hip Replacement (THR). In some instances, following resurfacing, further deterioration of the articular cartilage of the femoral head or the acetabulum eventually leads to a need for THR procedure.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

According to an aspect of the invention there is provided a joint distractor, including a distracting portion configured to accommodate a groin area of a subject, a stationary portion configured to immovably accommodate an ipsilateral leg of the subject, and an extension mechanism immovably coupled to the stationary portion and configured to distract and urge the distracting portion against the groin area.

According to an aspect of the invention there is provided a joint distractor, including a stationary portion configured to restrain movement of a body limb of a subject, a extractable portion shaped to accommodate a joint body area joining the limb with the body of the subject and an extension mechanism coupled to said stationary portion and configured to retract and/or distract and urge said extractable portion against said joint body area. The term “distract” as used herein means separating between articular surfaces of bones in a joint and forming a gap between the surfaces.

In some embodiments, the joint distractor is composed of materials compatible with at least an MRI scanner. In some embodiments, the joint distractor can be fitted for use on MRI and/or CT beds. The joint distractor is self-contained. The joint distractor is non-attachable to an orthopedic table.

According to an aspect of the invention there is provided a joint distractor that is portable. The joint distractor is used during surgery.

According to an aspect of the invention there is provided a method for scanning the articulate surfaces of the acetabulofemoral joint, including an extension of the joint of the acetabulofemoral joint using the joint distractor, and scanning the articulate surfaces using a scanner, e.g. MRI, CT.

According to an aspect the invention there is provided a method for separation of the pelvic girdle from the acetabulofemoral joint, including immobilizing a leg, and applying pressure at least to the left or right groin proximal to said leg.

According to an aspect the invention there is provided a method for a surgical THR procedure with an anterior approach to the acetabulofemoral joint, including applying positive pressure to the ipsilateral groin at the base of the leg to rotate the pelvic girdle about the acetabulofemoral joint of the contralateral leg, and a surgical THR procedure with an anterior approach.

According to an aspect of the invention there is provided a system for actuating a joint distractor, including a joint distractor, a means of measurement for at least one of strain, torque, pressure, or length, and a computer.

According to an aspect the invention there is provided a system for abduction and adduction of the knees, including an expansion device, whereby the expansion device expands towards an ipsilateral knee to apply a direct abducting force; and a band, whereby the band secures the contralateral knee onto the expansion device, thereby during expansion of the expansion device an indirect adduction force is applied to the contralateral knee by the expansion device balloon.

According to an aspect of the invention there is provided a method for positioning a Valgus knee, including positioning an expansion device facing the ipsilateral knee, and expanding the expansion device towards the ipsilateral knee.

According to an aspect of the invention there is provided a method for positioning a Varus knee, including securing a band to an expansion device facing the ipsilateral knee, securing the band to the contralateral knee, and expanding the expansion device.

According to an aspect the invention there is provided a system for abduction and adduction of the knees, and the system for abduction and adduction of the knees is self-contained.

According to an aspect of the invention there is provided a joint distractor, including: a stationary portion configured to fix a first body portion on a first side of a body joint, and an extendable portion configured to be urged against a second body portion on a second side of the body joint, and wherein extension of the extendable portion urges the extendable portion onto the second body portion away from the first body portion thereby forming a gap between articulating surfaces of the first and second body portions within the joint.

In some embodiments, the device is composed of materials compatible with at least an MRI scanner. In some embodiments, the distractor is configured to effect rotational distraction force on the second body portion in relation to the first body portion coupled by the joint to be distracted. In some embodiments, at least a portion of the extendable portion is angled/tilted so that to effect rotational distraction force on the second body portion in relation to the first body portion coupled by the joint to be distracted.

In some embodiments, the device is fitted for use on MRI and/or CT beds. In some embodiments, the device is self-contained. In some embodiments, the device is non- attachable to an orthopedic table. In some embodiments, the device is portable. In some embodiments, the device is configured to be used during surgery.

In some embodiments, the extractable portion including an extension mechanism including at least one of a telescopic extension mechanism, a scissors extension mechanism, a crank extension mechanism. In some embodiments, the extendable portion adjusts automatically. In some embodiments, the at least one of a strain gauge, a linear encoder, a pressure sensor, a proximity sensor, and an optical sensor. In some embodiments, the stationary portion includes a support member configured to maintain the limb stationary during the extension.

In some embodiments, the stationary portion includes a retainer configured to maintain the limb stationary in relation to the stationary potion. In some embodiments, at least one balloon coupled to the extendable portion and configured to expand away from the extendable portion. In some embodiments, the extender includes a pedal coupled to the extendable portion such that applying pressure to the pedal adjusts the distance between the pushing end and the stationary portion.

According to an aspect of the invention there is provided a method for separation of the pelvic girdle from the acetabulofemoral joint, including, immobilizing a leg, applying positive pressure at least to the left or right groin proximal to the leg.

According to an aspect of the invention there is provided a system for automatic joint distraction, including, a joint distractor including a stationary portion configured to fix a first body portion on a first side of a body joint, and an extendable portion configured to be urged against a second body portion on a second side of the body joint, and wherein extension of the extendable portion urges the extendable portion onto the second body portion away from the first body portion thereby forming a gap between articulating surfaces of the first and second body portions within the joint, at least one sensor, and a computer configured to adjust the distance between the pushing end and the stationary portion.

In some embodiments, the sensor is at least one of a strain gauge, a linear encoder, a pressure sensor, a proximity sensor, and an optical sensor. In some embodiments, the extendable portion includes an extension mechanism and the extension mechanism is controllable via the computer.

According to an aspect of the invention there is provided a knee positioning device, including, a stationary portion configured to fix a position of a leg of a subject, a balloon configured to expand away from the stationary portion such that the expansion applies an abducting force to an ipsilateral knee. In some embodiments, the device including a band configured to secure a contralateral knee of the subject onto the stationary portion such that expansion the balloon applies an indirect adduction force to the contralateral knee.

According to an aspect of the invention there is provided a method for positioning a knee in a valgus position, including, fixing a portion of a leg to a stationary portion of a device including a stationary portion configured to fix a position of a leg of a subject, and a balloon configured to expand away from the stationary portion, and applying an abducting force to an ipsilateral knee of the subject by expanding the balloon.

According to an aspect of the invention there is provided a method for positioning a knee in a varus position, including, fixing a portion of a leg to a stationary portion of a device including a stationary portion configured to fix a position of a leg of a subject, a balloon configured to expand away from the stationary portion, and a band configured to secure a contralateral knee of the subject onto the stationary portion, securing the band to the contralateral knee, and applying adduction force to the contralateral knee by expanding the balloon. In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A and 1B are side view simplified illustrations of an exemplary joint distractor, according to some embodiments of the present disclosure;

FIGS. 2A, 2B, 2C, and 2D are side view simplified illustrations of an exemplary joint distractor during implementation, according to some embodiments of the present disclosure;

FIGS. 3A and 3B are side view simplified illustrations of an exemplary embodiments of the joint distractor with scissors extension mechanism, according to some embodiments of the present disclosure;

FIGS. 4A and 4B are side view simplified illustrations of an exemplary embodiments of the joint distractor with telescope extension mechanism, according to some embodiments of the present disclosure;

FIGS. 5A and 5B are side view simplified illustrations of an exemplary embodiments of the joint distractor with crank extension mechanism, according to some embodiments of the present disclosure;

FIGS. 6A and 6B are side view simplified illustrations of an exemplary embodiments of the joint distractor with screw extension mechanism, according to some embodiments of the present disclosure;

FIG. 7 is a side view simplified illustration of an exemplary embodiment of the joint distractor with two foot-holders, according to some embodiments of the present disclosure;

FIG. 8 is a side view simplified illustration of an exemplary embodiment of the joint distractor with a leg clamp, according to some embodiments of the present disclosure;

FIGS. 9A, 9B, and 9C are side view simplified illustrations of an exemplary embodiments of the joint distractor with a rotation hinge, according to some embodiments of the present disclosure;

FIGS. 10A, 10B, 10C and 10D are side view simplified illustrations of exemplary embodiments of a pusher, according to some embodiments of the present disclosure;

FIG. 11 is a flow chart illustrating a method of scanning articulation surfaces of the femoral heal and acetabulum of an AFJ, according to some embodiments of the present disclosure;

FIG. 12 is a side view simplified illustration of a system for the extension of the joint distractor, according to some embodiments of the present disclosure; and

FIGS. 13A, 13B and 13C are side view simplified illustrations of a system for abduction and adduction of the hips and knees, according to some embodiments of the present disclosure.

### DETAILED DESCRIPTION

Orthopedic operations involving the AcetabuloFemoral Joint (AFJ) e.g., resurfacing of the articular surfaces, require observation of the surfaces of the articular cartilage covering the femoral head or the acetabulum. The AFJ is a ball-and socket type of joint, where the femoral head is surrounded by the acetabulum, and the bones are constantly in contact. In common practice, in order to observe the articular cartilage that covers the femoral head or the acetabulum, the AFJ is distracted during surgery for direct visual observation (e.g., by a surgeon).

Resurfacing techniques commonly cover or replace damaged articular surfaces with artificial articular surfaces. A technique such as that disclosed in PCT Patent Application Publication No. WO2018020490, to the same inventor, involves a resurfacing procedure in which the natural cartilage is covered by artificial articular surfaces. In this technique, the cartilage is covered using individually fitted surfaces which are printed for a specific surface, without the removal of natural tissue. The topography of the articular surfaces to be covered can be obtained using data collected by a scan, which can be obtained by one or more of MRI scanner and CT scanner. Therefore, it is a prerequisite that the articulate surfaces of the damaged cartilage can be scanned.

Although the scans of the articulate surfaces of many different joints can be obtained using MRI or CT scanners, scans of the femoral head or the acetabulum are challenging to produce due to the position of the femoral head which is surrounded by the acetabulum socket. The unique anatomy of the AFJ necessitates distancing the femoral head and acetabulum from each other to allow a scan of the articular surfaces.

Visual access of the AFJ (direct or indirect) is commonly obtained by extension of the AFJ and examination of the articulate surfaces. It is common to use a device such as a hip distractor in order to create a cavity between the femoral head and the acetabulum. Currently, scanning of the AFJ is limited. The commonly used hip distractors must attach to an orthopedic table for the extension process and are therefore unsuitable for use in conjunction with an MRI bed or CT bed. Additionally, the commonly used hip distractors are usually made of materials that are incompatible with MRI scanners. Commonly used hip distractors distract the AFJ by using a pelvic retaining element such as a padded cylinder, placed against the pelvis between the legs of a subject, along with a boot on the leg being distracted. The padded cylinder counters tension forces applied to the body by axial pulling of the leg to distract the joint. Thus, the femoral head is pulled away from the immobilized pelvis.

As disclosed in U.S. Pat. No. 6,035,227, to the same inventor, during the extension of the joint space, the pressure inside the joint space decreases, which creates a physiological effect known as the vacuum phenomenon. The decrease of pressure draws nitrogen from the surrounding tissue to equalize the pressure, which allows the expansion of the space to continue. It has been found that nitrogen gas can improve the contrast of the MRI images, by becoming a contrast agent for the MRI scans.

Additionally, the need to attach commonly used hip distractors to orthopedic tables creates a limitation of procedures involving hip extension, limiting such procedures to ORs. A potential advantage of hip distractors that can be used outside of an OR is in that such distractors allow greater availability of the OR to other procedures. Increasing OR availability and utilization improves hospital efficiency and budget. According to an aspect of some embodiments of the invention there is provided a joint distractor configured to distract a joint by expanding a distance between articular surfaces of a first and a second body portions coupled by the joint. In some embodiments, the distractor comprises a stationary portion and a distracting portion. In some embodiments, the distracting portion is configured to accommodate a groin area of a subject. In some embodiments, the stationary portion is configured to immovably accommodate an ipsilateral leg of said subject. In some embodiments, the joint distractor comprises an extension mechanism that is immovably coupled to said stationary portion and configured to distract and urge the distracting portion against the ipsilateral groin area. During the extension of the extension mechanism, asymmetric positive pressure is applied to the pelvic gridle at the ipsilateral groin. The asymmetrical positive pressure forces the rotation of the pelvic gridle about the contralateral AFJ while keeping both feet of the subject leveled on the same plane, thus distracting the distance between the femoral head and the acetabulum of the ipsilateral AFJ. As described in greater detail elsewhere herein, in some embodiments, the distractor can be employed to distract joints other than the AFJ e.g., the shoulder joint.

According to an aspect of some embodiments of the invention there is provided a joint distractor that is MRI or CT compatible. In some embodiments, the joint distractor is composed of materials compatible with at least an MRI imager. In some embodiments, the device is self-contained, and is configured to be used on an orthopedic and non-orthopedic table e.g., on an MRI table and/or a CT table.

According to an aspect of some embodiments of the invention there is provided a joint distractor that is self-contained (i.e., does not require attachment to, or support from an external object e.g., a table). In some embodiments, the distractor is configured to be coupled to two or more body portions sharing a joint. In some embodiments, the distractor is coupled at a first end to a first body portion on a first side of a joint, and at a second end to a second body portion on an opposite side of the joint.

In some embodiments, the distractor is expandable. In some embodiments, the distractor comprises a stationary portion and an extendable portion. In some embodiments, the stationary portion is coupled to a first body portion (e.g., a limb) on a first side of a joint and the extendable portion is coupled to a second body portion on a second side of a joint. In some embodiments, extension forces applied to a first body portion on one side of a joint are countered by countering forces generated by a second body portion on an opposite side of the joint.

For example, in some embodiments, a second body portion may be a subject's limb that counters tensile pressure applied to a first body portion e.g., the pelvic girdle by the joint distractor. In some embodiments, the joint distractor is configured to be used in a non-surgical, e.g. operating room (OR) environment. In some embodiments, the joint distractor is configured to be used on a surgery table without attachment to the table or to additional equipment. In some embodiments, the joint distractor is configured to be used in conjunction with MRI or CT tables.

According to an aspect of some embodiments of the invention the joint distractor is portable.

According to an aspect of some embodiments of the invention, there is provided a method for extension of the AFJ. In some embodiments, the method comprises immobilizing a leg. In some embodiments, the method comprises applying positive pressure to the ipsilateral groin at the base of the leg. In some embodiments, the method comprises rotating the pelvic girdle about the AFJ of the contralateral leg distancing the pelvic girdle acetabulum from the femoral head.

According to an aspect of some embodiments of the invention, there is provided a method for scanning the articulation surfaces of AFJ. In some embodiments, the method comprises coupling the device to a subject positioned on an MRI or CT table. In some embodiments, the method comprises applying positive pressure to the ipsilateral groin at the base of the leg to rotate the pelvic girdle about the AFJ of the contralateral leg. In some embodiments, the method comprises applying positive pressure to the ipsilateral groin at the base of the leg during the scan of at least an MRI or CT scanner.

According to an aspect of some embodiments of the invention, there is provided a joint distracting system comprising a joint distractor, a means of measurement of at least one of strain, torque, and/or length of extension, and a computer. In some embodiments, the system comprises one or more of a strain gauge, a pressure sensor, a proximity sensor, and an optical sensor. In some embodiments, the system is configured, for each extension of the joint distractor, based on input from one or more of the sensors to automatically limit one or more of strain, torque, and/or length of extension.

According to an aspect of some embodiments of the invention, there is provided a method for a surgical THR procedure with an anterior approach to an AFJ. In some embodiments, the method comprises applying positive pressure to the ipsilateral groin at the base of a leg to rotate the pelvic girdle about the AFJ of the contralateral leg. In some embodiments, the method comprises applying positive pressure to the ipsilateral groin at the base of the leg during a surgical THR procedure. In some embodiments, the method comprises a surgical THR procedure with an anterior approach. In some embodiments, the method comprises using the joint distractor in surgical procedures on an outpatient basis. In some embodiments, the method comprises using the joint distractor in procedures outside of an OR.

According to an aspect of some embodiments of the invention, there is provided a system for distraction of a joint, e.g., the hip joint, with concurrent abduction or adduction of the hips and/or knees. In some embodiments, the system comprises an expandable mechanism, e.g., a balloon. In some embodiments, the expandable mechanism is configured, when expanded to apply an abducting or adducting force onto a body portion e.g., a knee of a subject. In some embodiments, the abduction of the ipsilateral knee allows adduction of the ipsilateral hip, and the adduction of the ipsilateral knee allows abduction of the ipsilateral hip.

According to an aspect of some embodiments of the invention, there is provided a method for positioning a Valgus knee. In some embodiments, the method comprises expanding a balloon towards the ipsilateral knee. In some embodiments, the method comprises applying an abducting force onto the ipsilateral knee by the expansion of the balloon.

According to an aspect of some embodiments of the invention, there is provided a method for positioning a Varus knee. In some embodiments, the method comprises securing the contralateral knee to the balloon using a band. In some embodiments, the method comprises expanding the balloon towards the ipsilateral knee. In some embodiments, the method comprises pulling the band towards the ipsilateral knee. In some embodiments, the method comprises applying an adducting force onto the contralateral knee by the band.

### Self-Contained Joint Distractor

In some embodiments, the joint distractor is used to distract the length of the distance between the femoral head and the acetabulum in an AFJ of a subject without attaching the joint distractor to any equipment e.g., a bed or table. In some embodiments, no additional equipment is necessary for the operation of the joint distractor. According to some embodiments, distract and as explained in greater detail elsewhere herein, the joint distractor is used without restricting the joint distractor to anything other than the subject, and/or without restricting the subject to anything other than the joint distractor. In some embodiments, the joint distractor is independent of an orthopedic table for operation. In some embodiments, the joint distractor is used under surgical drape during operation. In some embodiments, the joint distractor is used during at least one of MRI or CT scans.

A potential advantage of the joint distractor is that it does not require an attachment to an orthopedic table for operation, therefore can be used on other table, e.g. surgical tables, as well as under surgical drapes during operation.

A potential advantage of using the joint distractor during surgical operations is that the joint distractor allows intraoperative scans.

### MRI Compatibility of the Joint Distractor

In some embodiments, the joint distractor is composed of materials compatible with at least a CT and/or MRI scanner. In some embodiments, the joint distractor is composed of materials which do not cause image disturbance in MRI scans. The joint distractor can contain any material that exhibits ferromagnetic qualities or any other material compatible with MRI, in composition, geometry, and/or quantity which may be used with MRI scanners. In some embodiments, the joint distractor is made of MRI and/or CT compatible materials. In some embodiments, the joint distractor can be made of one or more of CT/MRI compatible materials e.g., HDPE, wood, carbon fiber and polymers. In some embodiments, the joint distractor is composed of only non-metallic materials. In some embodiments, the joint distractor is self-contained, and operates independently of specialized tables, e.g., orthopedic tables. In some embodiments, joint distractor fits and operates on an MRI bed, and/or within an MRI scanner. In some embodiments, the joint distractor is self-contained, fits in an MRI machine, and composed of MRI compatible materials.

A potential advantage of the composition of joint distractor, which is compatible with at least a CT scanner, is that the joint distractor can be used to scan the articulate surfaces of the femoral head and/or the acetabulum using at least a CT and/or an MRI.

A potential advantage of the joint distractor being made of MRI and/or CT compatible materials is that the device can be used during scans of the articulate surfaces of the femoral head and/or the acetabulum using MRI or CT, which allows 3D-printing of new surfaces for the AFJ.

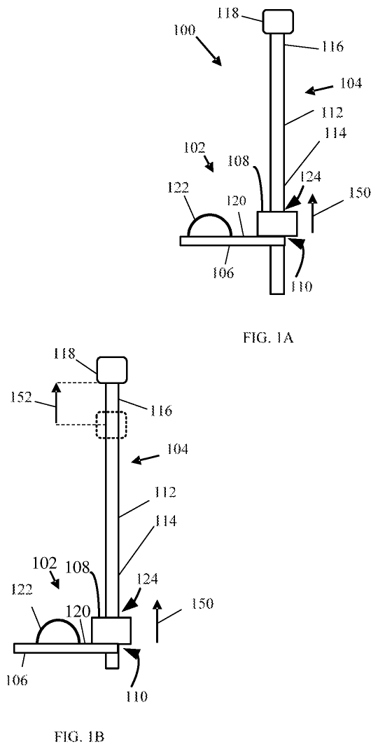

FIG. 1A which is a cross-section view simplified illustration of a joint distractor in accordance with some embodiments of the invention shows a joint distractor 100 comprising at least a stationary portion 102 and a distracting portion 104. The stationary portion 102 comprises a support member, for example, e.g., a foot rest 106. In some embodiments, the stationary portion 102 comprises an extension mechanism 108. The stationary portion 102 and extendable portion 104 are coupled via the extension mechanism 108. In some embodiments, the extendable portion 104 is perpendicular to the stationary portion. In some embodiments, the foot rest 106 is coupled to the extension mechanism 108.

In some embodiments, the foot rest 106 comprises at least one rigid base. In some embodiments, the foot rest 106 comprises a base. In some embodiments, the length and/or width of foot rest 106 ranges between 5 cm to 40 cm. In some embodiments, the length and/or width of the foot rest 106 is smaller than 5 cm or larger than 40 cm.

In some embodiments, the thickness of the foot rest 106 can range between 5 mm to 5 cm, and in some embodiments the depth of the foot rest 106 is smaller than 5 mm or larger than 5 cm. In some embodiments, the surface 120 of the foot rest 106 is flat. In some embodiments, the surface 120 of the foot rest 106 includes depressions and peaks as desired. In some embodiments, the surface 120 of the foot rest 106 is rigid.

The foot rest 106 can include at least one retainer 122 configured to maintain the position of a leg of a subject stationary. In some embodiments, the retainer 122 comprises at least one leg-fastening device. In some embodiments, the retainer 122 comprises of straps. In some embodiments, the retainer 122 is composed of flexible materials, semi-rigid materials, and rigid materials or any combination thereof. In some embodiments, the retainer 122 is adjustable and configured to accommodate a foot of a subject. In some embodiments, and as explained in greater detail elsewhere herein, the retainer 122 is adjustable using an electronic controller, such as, for example, a remote or a computer. In some embodiments, the retainer 122 is automatically adjusted by an electronic device.

In some embodiments, the foot rest 106 comprises at least one opening through which a foot of a subject can be placed. In some embodiments, the foot rest 106 is configured to restrict movement of the foot of the subject. In some embodiments, the foot rest 106 comprises a boot. In some embodiments, the boot is in the form of an orthopedic walker boot.

It is noted that the support member is described herein as foot rest 106, and it noted that the support member can be used to support any limb of the body, for example, an arm or a hand.

In some embodiments, and as described in greater detail elsewhere herein, the foot rest 106 comprises a top surface 120. In some embodiments, the top surface 120 is positioned to be facing a foot of a subject once the distracting portion is positioned between the legs of the subject.

In some embodiments, the foot rest 106 is rotatable about the axis parallel to the surface 120 of the foot rest 106. In some embodiments, the rotation and/or the adjustment of the foot rest 106 is controllable electronically, for example, by a user or a computer program. In some embodiments, the rotation and/or the adjustment of the foot rest 106 is automatic.

In some embodiments, the foot rest 106 and extension mechanism 108 are coupled at a base coupling 110. In some embodiments, the base coupling 110 is an integral part of either the foot rest 106, the extension mechanism 108, or both. In some embodiments, the base coupling 110 includes a pin which allows the connection and disconnection of the foot rest 106 from the extension mechanism 108. In some embodiments, the pin acts as a lock to temporarily set the foot rest 106 and extension mechanism 108 in place until the pin is released, which can unlock and/or disconnect the foot rest 106 from the extension mechanism 108. In some embodiments, the base coupling 110 has a lock and key configuration, for example, in some embodiments, the foot rest 106 and the extension mechanism 108 have corresponding geometries that interlock.

In some embodiments, the base coupling 110 comprises a screw mechanism comprising a threaded shaft and a threaded hole. In some embodiments, the foot rest 106 comprises a threaded hole through which the threaded shaft can screwed. In some embodiments, the extension mechanism 108 comprises of a threaded shaft fitted to screw through the foot rest 106. In some embodiments, and as described in greater detail elsewhere herein, the extendable portion 104 comprises the threaded shaft configured to screw fit through the foot rest 106.

In some embodiments such as the embodiment depicted by FIG. 1A, joint distractor 100 includes one or more extension mechanism 108. In some embodiments, the extension mechanism 108 is distractable in at least a direction parallel to the leg of the subject, for example, as depicted by an arrow 150. In some embodiments, the extension mechanism 108 distracts linearly. As explained elsewhere herein, the extension mechanism 108 is coupled to a pushing rod 112 having a pushing end 116.

In some embodiments, the extension mechanism 108 distracts the pushing end 116. In some embodiments, the extension mechanism 108 is distractable by up to 5 cm. In some embodiments, the extension mechanism 108 is distractable by more than 5 cm. In some embodiments, the extension of the extension mechanism 108 is distractable in increments of 0.05 mm. In some embodiments, the extension of the extension mechanism 108 is distractable in increments of 0.02 mm. In some embodiments, the length of the extension increments of the extension mechanism 108 is selected by a user and/or software.

In some embodiments, the extension mechanism 108 comprises a rack and pinion driving system. In some embodiments, the extension mechanism 108 comprises a telescoping driving system. In some embodiments, the extension mechanism 108 comprises a scissors extension mechanism. In some embodiments, the extension mechanism 108 comprises a threaded bore. In some embodiments, the pushing rod 112 comprises a threaded portion of configured to fit the threaded bore. In some embodiments, the pushing rod 112 is configured to be driven back and forth by rotation within the extension mechanism 108 threaded bore.

In some embodiments, the extendable portion 104 comprises a pushing rod 112 configured to support a pusher 118. In some embodiments, the pusher 118 is configured to apply positive pressure onto the ipsilateral groin area of a subject. The pushing rod 112 has a pushing end 116 and a countering end 114. In some embodiments, the stationary portion 102 and/or the extension mechanism 108 are coupled to the countering end 114. In some embodiments, the pusher 118 rests on the pushing end 116 of the pushing rod 112. In some embodiments, the pusher 118 is coupled to the pushing end 116.

In some embodiments, at least a portion of the pushing rod 112 is hollow. In some embodiments, at least a portion of the pushing rod 112 is solid. In some embodiments, the pushing rod 112 has an outer diameter of 3-7 cm. In some embodiments, the outer diameter of the pushing rod 112 is smaller than 3 cm or larger than 7 cm. In some embodiments, the outer diameter of the pushing rod 112 varies throughout the pushing rod 112. In some embodiments, the pushing rod 112 has a non-circular transverse cross section e.g., rectangular. In some embodiments, the length of pushing rod 112 is between 20-90 cm long. In some embodiments, the pushing rod is shorter than 20 cm. In some embodiments, the pushing rod 112 is longer than 90 cm.

In some embodiments, the pusher 118 is rigidly coupled to the pushing rod 112. In some embodiments, the pusher 118 is an integral part of the pushing rod 112. In some embodiments, the pusher 118 has a proximal surface and a distal surface in relation to the pushing rod 112. In some embodiments, the pusher 118 is attached to the pushing rod 112 by contact with at least the proximal end of the pusher 118. In some embodiments, the pusher 118 is elongated in a direction perpendicular to the longitudinal axis of the pushing rod 112.

In some embodiments, the pusher 118 extends from the pushing rod 112. In some embodiments, the pusher 118 comprises a projection extending from said extendable portion and configured to urge against a body of the subject.

In some embodiments, the pusher 118 comprises padding to at least its distal end, although in some embodiments the pusher 118 includes padding on any of its other surfaces along its longitudinal axis. In some embodiments, the pusher 118 comprises a surface having a plane normal to the longitudinal axis of the pushing rod 112. In some embodiments, the surface has a length and a width of 12 cm by 5 cm, respectively. In some embodiments, the length of the of the pusher 118 ranges between 7 cm to 15 cm. In some embodiments, the length of the of the pusher 118 is smaller than 7 cm or larger than 15 cm.

In some embodiments, the width of the of the pusher 118 ranges between 3 cm to 7 cm. In some embodiments, the width of the of the pusher 118 is smaller than 3 cm or larger than 7 cm. In some embodiments, the width and/or length of the pusher 118 are adjustable.

In some embodiments, the height of the pusher 118, in the direction of the longitudinal axis of the pushing rod 112 ranges between 2 cm to 5 cm. In some embodiments, the height of the pusher 118 in the direction of the longitudinal axis of the pushing rod 112 is smaller than 2 cm or larger than 5 cm.

In some embodiments, the extension mechanism 108 is coupled to the pushing rod 112 by an extension coupling 124. In some embodiments, the extension coupling 124 is an integral part of one or more of the extension mechanism 108 and the pushing rod 112. In some embodiments, the extension coupling 124 includes a pin positioned such that the pushing rod 112 is removably coupled to the extension mechanism 108. For example, in some embodiments, removal of the pin detaches the pushing rod 112 from the extension mechanism 108. In some embodiments, the pin acts as a lock to temporarily set the pushing rod 112 and extension mechanism 108 in place until the pin is released, which can unlock and/or disconnect the pushing rod 112 from the extension mechanism 108.

In some embodiments, the extension coupling 124 fixes the position of the extension mechanism 108 in relation to the pushing rod 112. In some embodiments, the extension coupling 124 fixed the position of the pushing rod 112 in relation to the extension mechanism 108.

In some embodiments, the extension coupling 124 comprises a screw mechanism which comprises a threaded shaft and a threaded hole. In some embodiments, the extension mechanism 108 comprises a threaded hole through which a threaded shaft can wind. In some embodiments, the pushing rod 112 comprises of a threaded shaft fitted to wind though the extension mechanism 108. In some embodiments, the extension coupling 124 includes a lock and key configuration, in which the extension mechanism 108 and the pushing rod 112 have interlocking geometries.

### Implementation of the Joint Distractor

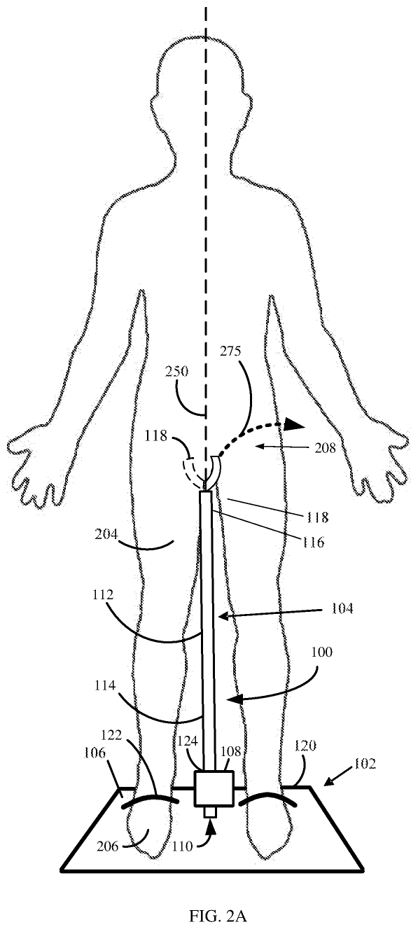

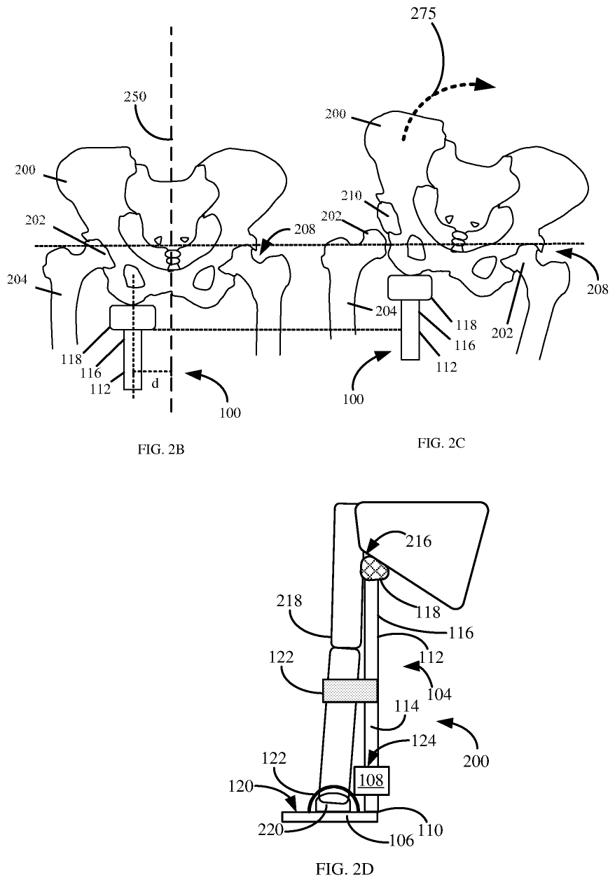

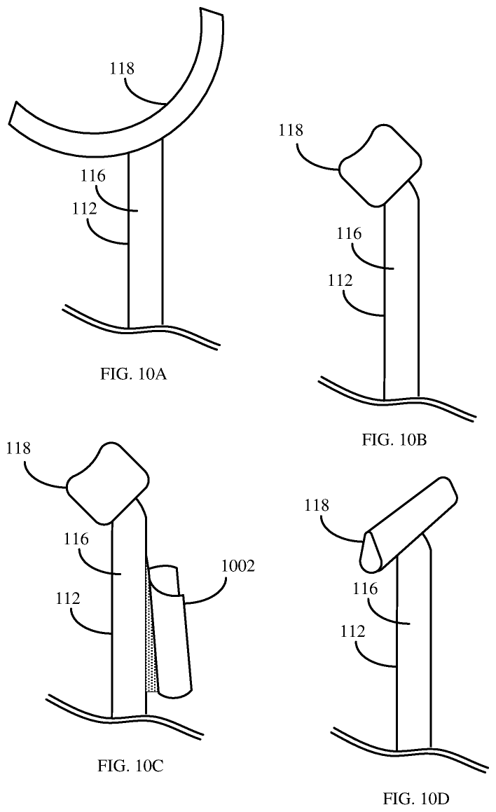

Reference is made to FIG. 2A-2D, which show side view simplified illustration of a joint distractor during implementation, according to some embodiments of the present invention. In some embodiments, the joint distractor 100 is configured to apply force to a body portion asymmetrically, e.g., at a distance (d) lateral or medial from the sagittal plane of the subject. In some embodiments, the joint distractor 100 is configured to apply force to a body portion asymmetrically, at a non-zero angle in relation to the sagittal plane of the subject. For example, in some embodiments, the joint distractor 100 is configured to apply asymmetric pressure to a groin of a subject in relation to a medial line 250 of the subject. As explained in greater detail elsewhere herein, in some embodiments, a portion of distractor 100, e.g., pusher 118, is set off-center or angled (e.g., FIGS. 10A-10D) so that to apply joint distracting force at a distance (d) lateral or medial from the sagittal plane of the subject.

In some embodiments, the joint distractor 100 is configured to be positioned such that the stationary portion 102 is coupled to a first body portion and the pushing end 116 and/or pusher 118 is urged against a second body portion. In some embodiments, the first and second body portions are disposed on opposite sides of a joint coupling articular surfaces of the portions to each other. In some embodiments, the first body portion is a limb and the second body portion is coupled to the first body portion by a joint.

In an exemplary embodiments depicted in FIGS. 2A, 2B, and 2C, the AcetabuloFemoral Joint (AFJ) is to be distracted by distractor 100, the first body portion is a leg of a subject and the second body portion is a pelvis of a subject. In an exemplary embodiments depicted in FIG. 2D, the glenohumeral joint (shoulder joint) is the joint to be distracted, the first body portion is an arm of a subject (Humerus) and the second body portion is a shoulder area (scapula) of the subject.

In some embodiments, the joint distractor 100 is configured to be positioned such that the pushing rod 112 is parallel to at least a portion of the first body portion (i.e., the limb). In some embodiments, the distance between the pusher 118 and the stationary portion 102 is adjustable, therefore, the first body portion of the subject can be fixed by the stationary portion 102 followed and the length of the joint extender can be placed between the first body portion and the second body portion and adjusted such that the pusher 118 is urged asymmetrically against the second body portion.

In some embodiments, and as described in greater detail elsewhere herein, the geometry of pusher 118 is shaped asymmetrically and configured to tend to move off-center in relation to the medial line 250. In some embodiments, the stationary portion 102 is configured to maintain the spatial orientation of the first body portion fixed in relation to the medial line 250.

In some embodiments, extension of the joint distractor distances the second body portion from the first body portion thereby distracting the joint between the body portions. In some embodiments, the pusher 118 is designed such that extension of the joint distractor exerts force onto the second body asymmetrically thereby changing (e.g., rotating) the spatial orientation of the second body portion in relation to the medial line 250. Therefore, the asymmetrical structure of distractor 100 brings about asymmetrical positioning of the distractor in respect to the body. As explained in greater detail elsewhere herein, the asymmetrical structure of distractor 100 and asymmetrical positioning of the distractor in respect to the body bring about rotation of the second body portion in respect to the first body portion. By fixing the first body portion and exerting rotating force onto the second portion, the joint distractor 100 distracts the joint between the first body portion and the second body portion. Therefore, in some embodiments, distractor 100 is configured to effecting rotational distraction force on a second body portion in relation to a second body portion coupled by the joint to distract the joint.

Reference is made to FIGS. 2A, 2B and 2C. In some embodiments, the subject is prostrate on the surface of a bed. In some embodiments, one ipsilateral limb, for example, a leg 204, of the subject is fixated by the footrest 106. In some embodiments, the foot rest 106 is set to the foot 206 or other areas of the leg 204. In some embodiments, restriction of movement of the leg can be achieved using the retainer 122, which can also be set on the foot 206 or other areas up to the groin at the leg, as desired.

The extension mechanism 108 is adjusted to fit the pusher 118 at a groin of the ipsilateral proximal end of the immobilized leg 204. For example, in some embodiments the extension mechanism 108 is distracted by a length depicted by an arrow 152. The extension mechanism 108 is then distracted to the desired length for joint extension, such that positive pressure is applied onto the pusher 118. The joint distractor 100 maintains the stationary leg 204 fixed in relation to the stationary portion 102 and/or the foot rest 106 such that the pressure applied onto the pusher 118 is countered by the stationary leg 204.

This countering force, along with the positive pressure applied to the pushing rod 118 by the extension mechanism 108, transfer the positive pressure to the pelvic gridle. Therefore, the positive pressure applied to the pelvic girdle 200 is asymmetrical and acting on the side of the pelvic girdle 200 corresponding with the stationary leg 204 which is immobilized. The asymmetrical positive pressure to the pelvic gridle 200 causes the pelvic gridle 200 to rotate about the AFJ 208 for example, e.g., in along at least a portion of the path indicated by an arced dotted arrow designated reference number 275.

The rotation of the pelvic gridle 200 in relation to the stationary leg 204 increases the distance between the femoral head 202 and the acetabulum of AFJ. The extension of the extension mechanism 108 continues distracting and/or applying positive pressure to the pelvic gridle 200 until sufficient pressure has been applied such that a desired distance between the femoral head 202 and the acetabulum 210 of AFJ is reached. In some embodiments, the desired distance between the femoral head and the acetabulum 210 of AFJ is that which allows visual access for 3D imaging of the articulate surfaces of the femoral head and the acetabulum.

In some embodiments, the joint distractor 100 is used to retract the pelvic girdle 200 away from the femoral head 202, thus distracting the length of the distance between the femoral head 202 and the acetabulum 210 within the AFJ in the following method: The foot rest 106 is used to immobilize the leg 204 while the device 100 is distracted to the ipsilateral groin at the base of the leg 212 proximal to the leg 204, using the extension mechanism 108. Throughout the extension procedure, the pushing end 116 exerts force onto the pusher 118 such in the direction parallel to the medial line 250 such that the pusher 118 transfers the positive pressure onto the pelvic girdle 200.

The pelvic gridle 200 counters the pressure applied by the pusher 118 for example, in a direction parallel to the medial line 250 and opposing the direction of the exerted force applied onto the pusher 118 by the pushing end 116. However, the stationary leg 204 immobilized and cannot retract; therefore, the extension of the device 100 forces the pelvic girdle 200 away from the immobilized femoral head 202 of the stationary leg 204.

In some embodiments, the pelvic gridle 200 is rotated about the axis of the AFJ of a non-immobilized leg 208. The rotation of the pelvic gridle 200 continues about the axis of the AFJ of the non-immobilized leg 208, while both feet of the subject can remain leveled on the same plane throughout the procedure. For simplicity of explanation, the effect of the spine on the pelvic girdle rotation is ignored.

Reference is made to FIG. 2D. According to some embodiments, the joint distractor 200/100 is used to distract a humerus bone of a subject from a clavicle of the subject. In some embodiments, the device 100 is implemented by positioning the joint distractor 100 at an axilla 216. In some embodiments, the arm 218 is abducted to 45 degrees. In some embodiments, the arm 218 is fixed using an arm retainer 122, which can be set to the hand 220 or other areas of the arm 218.

The extension mechanism 108 is distracted or contracted to place the pusher 118 at the axilla 216. The extension mechanism 108 is then distracted to the desired length for joint extension, which applies positive pressure onto the pusher 118, which is transferred to a body portion. The stationary arm 218 is maintained immobilized in relation to the stationary portion 102, thus, a countering force is applied back onto the retainer 122 from the stationary arm 218. This countering force, along with the positive pressure applied to the pusher 118 by the extension mechanism 108, acts on the ipsilateral scapula. Therefore, the pusher 118 applies positive pressure to the ipsilateral scapula.

The positive pressure applied by the pusher 118 to the ipsilateral scapula pushes the ipsilateral scapula away from the Humerus, thereby increasing the distance between the humeral head and the glenoid fossa. The extension of the extension mechanism 108 continues until sufficient pressure has been applied such that a distance between the humeral head and the glenoid fossa has reached a desired length. For example, in some embodiments, the desired length of distance between the humeral head and the glenoid fossa is that which allows visual access for 3D imaging of the articulate surfaces of the humeral head and the glenoid fossa.

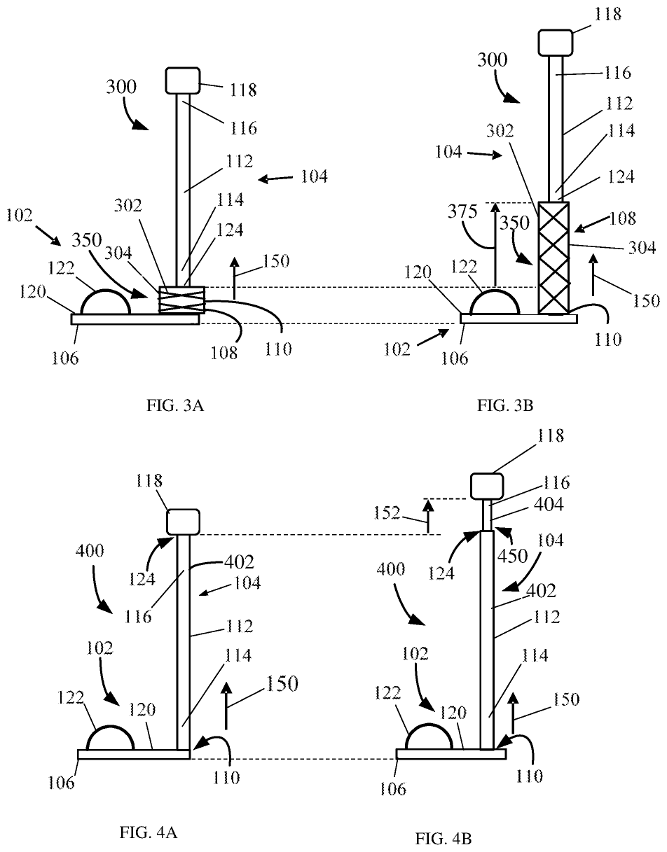

Reference is made to FIG. 3A-3B, which are side view simplified illustrations of an exemplary embodiments of the joint distractor with scissors extension mechanism, according to some embodiments of the present disclosuredistract, and to FIG. 4A-4B, which are side view simplified illustrations of an exemplary embodiments of the joint distractor with telescope extension mechanism, according to some embodiments of the present di sclosuredistract.

In some embodiments of the joint distractor 100/200/300/400, such as depicted in FIG. 3A-3B, the extension mechanism 108 comprises a scissors extension mechanism 350. In some embodiments, the scissors extension mechanism 350 is distractable in the direction of arrow 150. In some embodiments, the scissors extension mechanism 350 includes linked crossing bars 302. In some embodiments can include a case 304 and/or sleeve to enclose the bars 302.

In some embodiments of the joint distractor 100/200/300/400, such as seen in FIG. 4A-4B, the extension mechanism 108 comprises of a telescope extension mechanism 450. In some embodiments, the telescope extension mechanism 450 includes one or more tubular sheaths 402. In some embodiments, the one or more tubular sheaths 402 are coaxial. In some embodiments the one or more tubular sheaths 402 are positioned inside and/or outside a pole 404. In some embodiments, the pole 404 is hollow. In some embodiments, the telescope extension mechanism 450 increases the length of the joint distractor 100/200/300/400 by shifting at least one tubular sheath 402 in relation to another tubular sheath 402 and/or the pole 404, for example, in the direction of arrow 150.

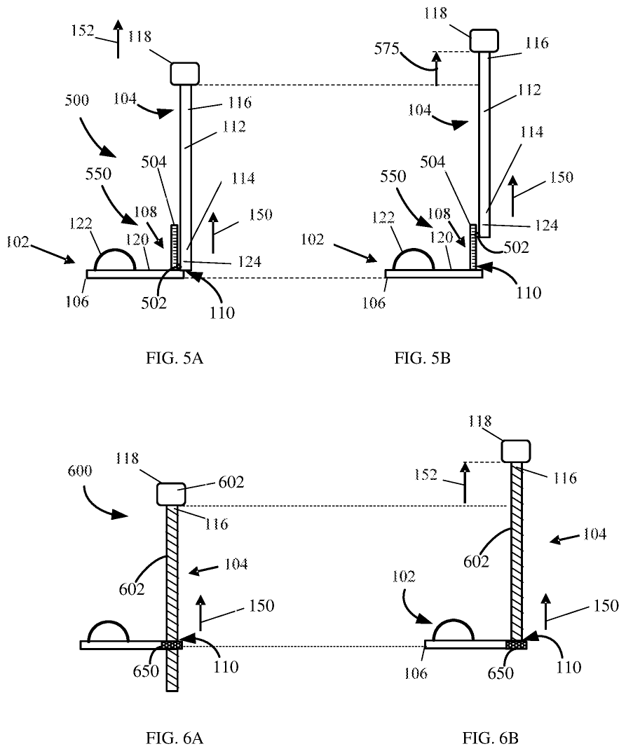

Reference is made to FIG. 5A-5B, which are side view simplified illustrations of an exemplary embodiments of the joint distractor with crank extension mechanism, according to some embodiments of the present disclosuredistract, and to FIG. 6A-6B, which are side view simplified illustrations of an exemplary embodiments of the joint distractor with screw extension mechanism, according to some embodiments of the present di sclosuredistract.

In some embodiments of the joint distractor 100/200/300/400/500/600, such as depicted in FIG. 5A-5B, the extension mechanism 108 includes a crank mechanism 550. In some embodiments, the crank mechanism 550 includes at least one gear 502 and at least one rail 504. In some embodiments, the gear 502 is linked to the extendable portion 104 distract and the rail 504 is coupled to the stationary portion 102. For example, in some embodiments, the rail 504 is coupled to the stationary portion 102 parallel to the distracting portion 104. In some embodiments, the rail 504 comprises creased increments which can fit the gear 502 and/or teeth of the gear 502. In some embodiments, rotation of the gear 502 changes the distance between the stationary portion 102 and the pusher 118.

In some embodiments, rotation of the gear 502 in relation to the extendable portion 104 changes the position of the gear 502 in relation to the rail 504 and/or repositions one or more gear 502 teeth from one increment of the rail 504 to another increment of the rail 504.

In some embodiments of the joint distractor 100/200/300/400/500/600, such as depicted in FIG. 6A-6B, and as described in greater detail elsewhere herein, the extension mechanism 108 comprises a screw mechanism 650 in which the stationary portion 102 comprises a threaded shaft 602, and the extendable portion 104 comprises one or more ridges 604 which are slidable through the threaded shaft 602. In some embodiments, the pusher 118 can include a similar threaded shaft. In some embodiments, rotation of one or more of the stationary portion 102 and the extendable portion 104 in relation to one another changes the distance between the stationary portion 102 and the pusher 118.

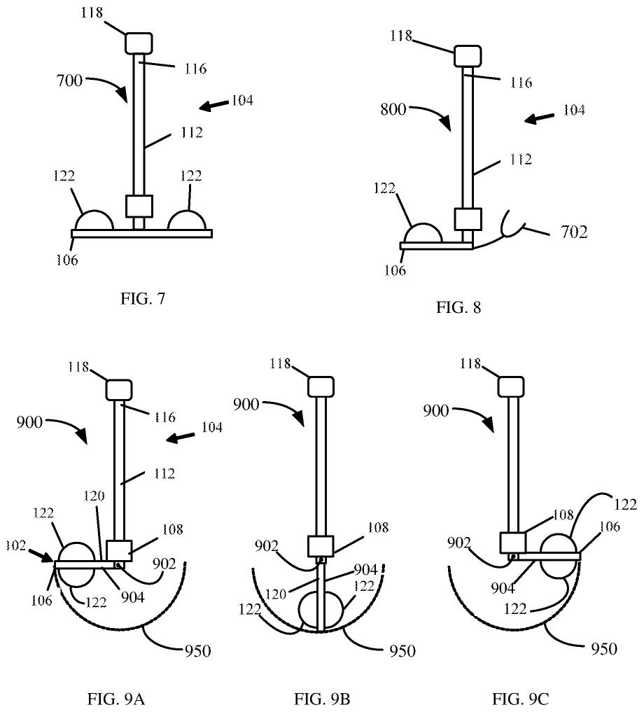

Reference is made to FIG. 7, which is a side view simplified illustration of an exemplary embodiment of the joint distractor with two foot-holders, according to some embodiments of the present disclosuredistract, and to FIG. 8, which is a side view simplified illustration of an exemplary embodiment of the joint distractor with a leg clamp, according to some embodiments of the present disclosuredistract.

In some embodiments, the joint distractor 100/200/300/400/500/600/700/800 can include a holder 702 configured to maintain a contralateral foot of the subject fixed in relation to the ipsilateral foot of the subject. In some embodiments, the holder 702 is configured to clasp the contralateral foot of a subject. In some embodiments, as depicted by FIG. 7, the holder 702 distracts from the stationary portion 102. In some embodiments, as depicted by FIG. 8, the holder 702 is removably coupled to the stationary portion 102. In some embodiments, the holder 702 is linked to the stationary portion 102. In some embodiments, the holder 702 comprises one or more retainers 122-7.

In some embodiments, the one or more retainers 122-7 positioned on the holder 702 are similar and/or symmetrical to the one or more retainers 122 positioned on the stationary portion 106. In some embodiments, the stationary portion 106 comprises the holder 702. In some embodiments, the stationary portion 106 and the holder 702 are leveled.

In some embodiments, the holder 702 comprises a clamp 802. In some embodiments, the clamp 802 is configured to maintain the position of the contralateral foot of the subject fixed in relation to the ipsilateral foot of the subject.

Reference is made to FIG. 9A-9C, which are side view simplified illustrations of an exemplary embodiments of the joint distractor with a rotation hinge, according to some embodiments of the present disclosuredistract. In some embodiments, the joint distractor 100/200/300/400/500/600/700/800/900 comprises a rotation hinge 902 about which the stationary portion 102 can rotate in relation to one or more of the rotation hinge 902, the extension mechanism 108, and the distracting portion 104.

In some embodiments, the base coupling 110 comprises the rotation hinge 902. In some embodiments, the rotation hinge 902 defines an axis of rotation about which the foot rest 106 is rotatable in relation to one or more of the extension mechanism 108, the hinge 902, and the distracting portion 104. In some embodiments, the rotational axis defined by the hinge 902 is parallel to an axis normal to the surface 120.

In some embodiments, the axis of rotation of the hinge 902 at base coupling 110 creates an acute or obtuse angle between the surface 120 and the extendable portion 104. In some embodiments, the hinge 902 allows movement of the foot rest 106 in relation to the extension mechanism 108. In some embodiments, the rotation axis defined by the hinge 902 is parallel to the surface of the foot rest 120. In some embodiments, the foot rest 106 includes a second retainer 129 on a surface 904 opposing the surface 120. In some embodiments, the second retainer 129 is similar to the retainer 122 as described elsewhere herein.

In some embodiments, the foot rest 106 is rotatable by at least 180 degrees e.g., along a path of travel 950. For example, in some embodiments, the foot rest 106 is rotatable from a position in which the retainer 122 is positioned facing the pusher 118, as depicted by FIG. 9A, to a position depicted by FIG. 9C, wherein the second retainer 129 is facing the pusher 118.

A potential advantage of the joint distractor 100/200/300/400/500/600/700/800/900 having a rotational axis defined by the hinge 902 is in that the joint distractor is usable for either the right or left foot of the subject without major adjustments to the position of the joint distractor in relation to the subject. for example, a joint distractor positioned between the legs of the subject can be adjusted to be used with a right foot or a left foot of the subject without removing the extendable portion 104 from between the legs of the subject.

extendable portion 104 Reference is made to FIGS. 10A, 10B, 10C and 10D, which are side view simplified illustrations of exemplary embodiments of a pusher, in accordance with some embodiments of the present disclosure. In some embodiments, the geometry of the pusher is designed to guide the pusher asymmetrically during extension, e.g., to one side of the groin of the subject. In some embodiments, the geometry of the pusher 118 is configured to fit a specific body portion of a subject, e.g., a groin. In some embodiments, the pusher 118 is structured to remain static.

In some embodiments, the pusher 118 is structured to accommodate a portion of the second body part of the subject such that a direction of the force exerted on the second body portion by the pusher 118 is fixed. In some embodiments, the pusher is shaped to accommodate a specific region of the joint to be distracted. E.g., in the embodiment depicted in FIG. 10A pusher 118 is shaped as a crescent so that to receive the groin area disposed medially over the hip joint to one side of the groin. In some embodiments, the pusher 118 is shaped asymmetrically. In some embodiments, the pusher 118 is geometrically shaped to correspond to an off-sagittal plane concave body portion.

In some embodiments, such as depicted by FIGS. 10B and 10C, the pusher 118 comprises a soft member, for example, a pillow, configured to accommodate the shape of the second body portion. In some embodiments, such as depicted by FIG. 10D, the pusher 118 is wedge-shaped to fit in the concavity formed in the groin over the medial side of the AFJ.

In some embodiments, the pusher 118 is coaxial with one or more of the distracting portion 104, the extension mechanism 108, and the pushing rod 112. In some embodiments, the pusher 118 is tilted in relation to the pushing rod 112. Therefore, the pushing rod 112 can be positioned coaxially with the medial line 250 while exerting force at a distance (d) lateral to the sagittal plane of the subject.

In some embodiments, the joint distractor 100 comprises a laterally countering element 1002. In some embodiments, the laterally countering element 1002 is configured to accommodate a contralateral limb (e.g., leg). In some embodiments, during extension of distractor 100, laterally countering element 1002 is configured to urge pusher 118 towards the contralateral joint thereby supporting asymmetrical distraction of the joint to be distracted. In some embodiments of the joint distractor 100/200/300/400/500/600/700/800/900/1000/1100/1200 the pusher 118 comprises an arc-shaped rod with a concave depression on the distal end. In some embodiments, the pusher 118 comprises one or more arms 1202 at one edge of the distal end of the pusher 118. In some embodiments, the one or more arms 1202 are coupled to the pusher 118.

In some embodiments, the one or more arms 1202 are elongated and/or straight. In some embodiments, the arm is parallel to the pushing rod 112. In some embodiments, the arm 1202 is angled in relation to the longitudinal axis of the pushing rod 112. In some embodiments, the arm 1202 is padded to accommodate the ipsilateral groin of a subject. In some embodiments, the one or more arms 1202 comprise adjustable segments which are couplable such that the angles between each segment are adjustable. In some embodiments, the adjustable segments are controlled electronically by a user and/or a software.

### Portability of the Joint Distractor

According to some embodiments, the joint distractor 100/200/300/400/500/600/700/800/900/1000/1100/1200 comprises a plurality of foldable segments which are couplable at one or more hinges. In some embodiments, the joint distractor 100/200/300/400/500/600/700/800/900/1000/1100/1200 comprises retractable segments. In some embodiments, the joint distractor can be taken apart and put together.

In some embodiments, the joint distractor 100/200/300/400/500/600/700/800/900/1000/1100/1200 is portable. In some embodiments, the joint distractor includes a carrying handle configured to carry the device. In some embodiments, the joint distractor comprises at least one wheel configured to support and/or transport the joint distractor. In some embodiments, joint distractor comprises a case in which at least one portion of the joint distractor can fit. In some embodiments, the joint distractor is configured to be lightweight, for example, by comprising one or more hollow component e.g. pushing rod 112. In some embodiments, the joint distractor is made of lightweight materials e.g., carbon fibers, aluminum alloys.

A potential advantage of the joint distractor 100/200/300/400/500/600/700/800/900/1000/1100/1200 is that it is portable and can be carried the to/from surgical rooms quickly.

A potential advantage of the joint distractor 100/200/300/400/500/600/700/800/900/1000/1100/1200 which can be used on any table is that hip operation procedures can be carried out on an outpatient basis, which increases OR availability, enables quick turnaround in hip procedures and reduces medical costs and hospital budget.

### Method of Scanning Articulation Surfaces Using the Joint Distractor

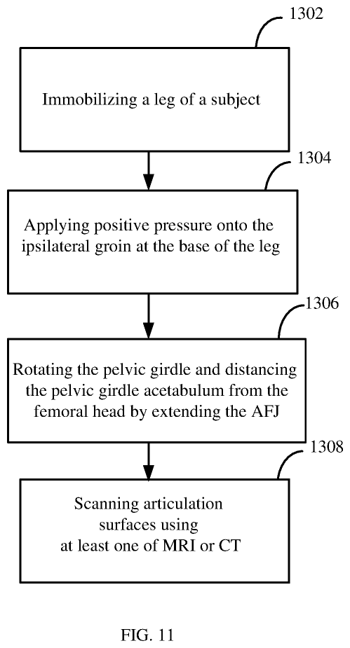

Reference is made to FIG. 11, which shows a flow chart illustrating a method of scanning articulation surfaces of the femoral heal and acetabulum of an AFJ, according to some embodiments of the present disclosure. In some embodiments, the method comprises coupling the device to a subject positioned on an MRI or CT table. In some embodiments, at step 1302, the method comprises immobilizing a leg.

In some embodiments, at step 1304, the method comprises applying positive pressure to the ipsilateral groin at the base of the leg to rotate the pelvic girdle about the AFJ of the contralateral leg. In some embodiments, at step 1306, the method comprises rotating the girdle and distancing the pelvic girdle acetabulum from the femoral head. In some embodiments, the expansion of the AFJ is done on a bed, and in some embodiments, the bed can fit inside at least one of MRI or CT scanners. In some embodiments, at step 1308, the method comprises scanning articulation surfaces using at least on of MRI or CT, after some the extension to the AFJ.

### System for Extension Using the Joint Distractor

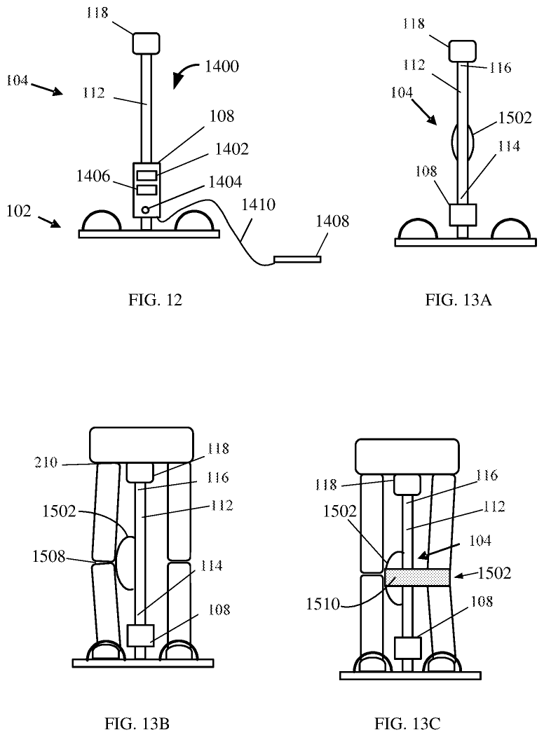

Reference is made to FIG. 12, which shows a side view simplified illustration of a system for the extension of the joint distractor, according to some embodiments of the present disclosure. In some embodiments, the system 1400 comprises a joint distractor 100/200/300/400/500/600/700/800/900/1000/1100/1200/1450, one or more sensors 1404 configured to measure one or more of strain, torque, pressure, or length of extension. In some embodiments, the one or more sensor 1404 comprises one or more of a strain gauge, a linear encoder, a pressure sensor, a proximity sensor, and an optical sensor.

In some embodiments, the strain detected by the one or more sensors 1404 is stain of one or more of the pushing rod 112, the pusher 118, the stationary portion 102, and the distracting portion 104. In some embodiments, the pressure detected by the one or more sensors 1404 is pressure applied to one or more of the pushing rod 112, the pusher 118, the stationary portion 102, and the distracting portion 104.

In some embodiments, the torque detected by the one or more sensors 1404 is torque applied to one or more of the pushing rod 112, the pusher 118, the stationary portion 102, and the distracting portion 104. In some embodiments, the length of extension detected by the one or more sensors 1404 is the distance between the stationary portion 102 and the pusher 118 before, during and/or after extension of the extension mechanism 108.

In some embodiments, the system 1400 comprises a computer 1402. In some embodiments, the one or more sensor 1404 is coupled to at least one portion of the joint distractor. In some embodiments, the one or more sensor 1404 is coupled to the joint distractor rigidly, by adhesion, by an electronic cable, by Wi-Fi, Bluetooth and/or the like. In some embodiments, the one or more sensors 1404 are in communication with the computer 1402.

In some embodiments, the computer 1402 controls the length of the extension of the extension mechanism 108 and/or the distance between the stationary portion 102 and the pusher 118 before, during and/or after extension of the extension mechanism 108. In some embodiments, the computer 1402 controls the starting and stopping operations of extension of the extension mechanism 108 distract.

In some embodiments, the total strain, torque, pressure, or length can be limited by a user. For example, in some embodiments, the system 1404 comprises a user interface module into which a user can input a limit for one or more of the strain, torque, pressure, or length such that the extension of the extension mechanism 108 is operated until one of the inputted limits are reached. In some embodiments, the limits of the strain, torque, pressure, or length are predetermined. In some embodiments, the limits of the strain, torque, pressure, or length are patient specific. distract

In some embodiments, the extension mechanism 108 is controlled by the computer 1402. in some embodiments, the computer 1402 is in communication with the user interface module.

In some embodiments the system 1400 comprises a pedal 1408 configured to control the extension of the extension mechanism 108. In some embodiments, the pedal 1408 is configured to grant an operator and/or user control of the extension mechanism 108. For example, in some embodiments, applying pressure to the pedal 1408 distracts and/or contracts the extension mechanism 108, or, in other words, increases and/or decreases the distance between the stationary portion 102 and the pusher 118.

In some embodiments, the pedal 1408 is coupled to the extension mechanism 108 by a data pathway 1410. In some embodiments, the data pathway 1410 comprises one or more of a tube, an electrical cable, WiFi, and Bluetooth. In some embodiments, the computer 1402 is in communication with one or more of the pedal 1408 and the data pathway 1410. distractdistract

In some embodiments, the system 1400 comprises control buttons. In some embodiments, the system comprises a display 1406 in communication with one or more of the pedal 1408 and the computer 1402. In some embodiments, the display 1406 is configured to display the measured quantities of at least one of strain, torque, or length. In some embodiments, the display 1406 comprises the user interface module.

In some embodiments, the system 1400 comprises an auditory sensor. In some embodiments, the system comprises a remote control. In some embodiments, the system is sound activated and/or controlled. In some embodiments, the system 1400 comprises machine learning algorithms for at least optimization of the joint extension. In some embodiments, the extension of the extension mechanism 108 is automatic.

A potential advantage of the one or more sensors 1404 and/or the display 1406 is in that the amount of stain, torque, and/or pressure that is applied to the subject is monitorable and controllable.

### Surgical Uses of the Joint Distractor

In some embodiments, and as explained in greater detail elsewhere herein, the joint distractor 100/200/300/400/500/600/700/800/900/1000/1100/1200/1450 is self-contained and operable by using a subject's own limb to counter urging pressure applied to the pelvic gridle on an ipsilateral side of the leg to distract the cavity within the ipsilateral AFJ of a subject. Hence, the joint distractor can be implemented on any surface (e.g., clinical table, surgical table, CT table, MRI table) independent of an orthopedic table.

A potential advantage in the self-contained joint distractor is in that it enables implementation of the joint distractor in outpatient procedures and OR operations. The joint distractor can be composed of materials compatible with at least an MRI scanner, hence, during surgical procedures involving the joint distractor, the patient can undergo intraoperative scanning using at least one of MRI or CT.

In some embodiments, distract there is provided a method for a surgical THR procedure with an anterior approach to the AFJ. In some embodiments, the method comprises applying positive pressure to the ipsilateral groin at the base of the leg to rotate the pelvic girdle about the AFJ of the contralateral leg. In some embodiments, the method comprises applying positive pressure to the ipsilateral groin at the base of the leg during a surgical THR procedure. In some embodiments, the method comprises a surgical THR procedure with an anterior approach.

A potential advantage of the joint distractor during surgical operations is that the joint distractor allows the exposure of the femur without the addition of specialized instruments or tables, while keeping the leg of the subject immobile, having the ability to prevent femoral fractures, during operations using the anterior approach.

### Abduction and Adduction of the Hips and Knees

Reference is made to FIG. 13A-13C, which show side view simplified illustrations of a system for abduction and adduction of the hips and knees, according to some embodiments of the present disclosure. In some embodiments, and as depicted by FIG. 13A, the system comprises at least one balloon 1502 positioned on the pushing rod 112 or otherwise elsewhere on the joint distractor 100/200/300/400/500/600/700/800/900/1000/1100/1200/1450/1500, such that the balloon 1502 accommodates a knee of a subject, having the ability to expand and contract.

In some embodiments, the position of the balloon 1502 is adjustable. In some embodiments, the expansion and/or contraction of the balloon 1502 are controllable, for example, by a user or computer. In some embodiments, the expansion and/or contraction of the balloon 1502 are controllable using an electronic device, such as, for example, a remote or user interface module.

In some embodiments, such as shown in FIG. 13B, the expansion of the balloon 1502 applies an abducting force onto the ipsilateral knee. In some embodiments, the balloon 1502 expands in at least the direction towards the ipsilateral knee 1508 of the subject. In some embodiments, the balloon 1502 expands to a volume sufficient to abduct the ipsilateral knee 1508.

In some embodiments, as depicted by FIG. 13C, the expansion of the balloon 1502 applies and adducting force onto the contralateral knee 1512. In some embodiments, a band 1510 secures the contralateral knee 1512 to the balloon 1502. In some embodiments, the expansion of the balloon 1502 pulls the band 1510 towards the ipsilateral knee 1508. In some embodiments, the band 1510 applies an adducting force onto the contralateral knee 1512. In some embodiments, the balloon 1502 expands to a volume sufficient to adduct the contralateral knee 1512.

### Method for Valgus Knee Position

According to some embodiments of the joint distractor there is provided a method for positioning a knee in a valgus position. In some embodiments, the method comprises expanding the balloon 1502 towards the ipsilateral knee 1508. In some embodiments, the method comprises applying an abducting force onto the ipsilateral knee 1508. In some embodiments, the method comprises applying an abducting force onto the ipsilateral knee 1508 by expanding the balloon 1502 to a desired volume.

In some embodiments, the method comprises expanding the balloon 1502 to a volume in which the ipsilateral knee is abducted to a valgus position. In some embodiments, the method comprises scanning of at least the ipsilateral knee 1508 before, during, and/or after the expansion of the balloon 1502. In some embodiments, the method comprises of scanning of at least the ipsilateral knee 1508 once the balloon 1502 has reached the desired volume.

### Method for Varus Knee Position

According to some embodiments of the joint distractor there is provided a method for positioning a knee in a varus position. In some embodiments, the method comprises expanding the balloon 1502 towards the ipsilateral knee 1508. In some embodiments, the method comprises securing the contralateral knee 1512 to the balloon 1502 using a band 1510. In some embodiments, the method comprises pulling the band 1510. In some embodiments, the method comprises expanding the balloon 1502 towards the ipsilateral knee 1508. In some embodiments, the method comprises applying an adducting force onto the contralateral knee 1512 during the expansion of the balloon 1502. In some embodiments, the band 1510 applies the adducting force onto the contralateral knee 1512.

In some embodiments, the method comprises of the band 1510 applying an adducting force onto the contralateral knee 1512 during and/or after expansion of the balloon 1502. In some embodiments, the method comprises expanding the balloon 1502 to a volume sufficient to abduct the contralateral knee 1512 such that the contralateral knee 1512 is positioned in a valgus position. In some embodiments, the method comprises scanning at least the contralateral knee 1512 before, during, and/or after the expansion of the balloon 1502. In some embodiments, the method comprises scanning at least the contralateral knee 1512 once the balloon 1502 has reached the desired volume.

A potential advantage of the adduction method and the abduction method is in that it can be used to control the angle of the femur during and after the extension of the AFJ. The angles of adduction and abduction of the knees and the hips are controllable using these methods, and no additional equipment or personal are needed to maintain the position of the bones fixed. Therefore, the subject can undergo scans during which the angles of the knees and hips are maintained in the desired positions.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A joint distractor, comprising:
a stationary portion configured to fix a first body portion on a first side of a body joint; and
an extendable portion configured to be urged against a second body portion on a second side of said body joint; and
wherein extension of said extendable portion urges said extendable portion onto said second body portion away from said first body portion thereby forming a gap between articulating surfaces of said first and second body portions within said joint.

2. The joint distractor according to claim 1, wherein the joint distractor is composed of materials compatible with at least an MRI scanner.

3. The joint distractor according to claim 1, wherein said joint distractor is configured to effect rotational distraction force on said second body portion in relation to said first body portion coupled by the joint to be distracted.

4. The joint distractor according to claim 1, wherein at least a portion of said extendable portion is angled/tilted so that to effect rotational distraction force on said second body portion in relation to said first body portion coupled by the joint to be distracted.

5. The joint distractor according to claim 1, wherein the joint distractor is fitted for use on MRI and/or CT beds.

6. The joint distractor according to claim 1, wherein the joint distractor is self-contained.

7. The joint distractor according to claim 1, wherein the joint distractor is non-attachable to an orthopedic table.

8. The joint distractor according to claim 1, wherein the joint distractor is portable.

9. The joint distractor according to claim I, wherein the joint distractor is configured to be used during surgery.

10. The joint distractor according to claim 1, wherein said extendable portion comprising an extension mechanism comprising at least one of: a telescopic extension mechanism, a scissors extension mechanism, and a crank extension mechanism.

11. The joint distractor according to claim 1, wherein said extendable portion adjusts automatically.

12. The joint distractor according to claim 1, comprising at least one of: a strain gauge, a linear encoder, a pressure sensor, a proximity sensor, and an optical sensor.

13. The joint distractor according to claim 1, wherein said stationary portion comprises a support member configured to maintain a limb stationary during said extension.

14. The joint distractor according to claim 1, wherein said stationary portion comprises a retainer configured to maintain a limb stationary in relation to said stationary potion.

15. The joint distractor according to claim 1, comprising at least one balloon coupled to said extendable portion and configured to expand away from said extendable portion.

16. The joint distractor according to claim 1, comprising a pedal coupled to said extendable portion such that applying pressure to said pedal adjusts a distance between a pushing end of said extendable portion and said stationary portion.

17. A method for separation of the pelvic girdle from the acetabulofemoral joint, comprising:
immobilizing a leg; and
applying positive pressure at least to the left or right groin proximal to said leg.

18. A system for automatic joint distraction, comprising:
a joint distractor comprising:
a stationary portion configured to fix a first body portion on a first side of a body joint; and
an extendable portion configured to be urged against a second body portion on a second side of said body joint; and
wherein extension of said extendable portion urges said extendable portion onto said second body portion away from said first body portion thereby forming a gap between articulating surfaces of said first and second body portions within said joint;
at least one sensor; and
a computer configured to adjust a distance between a pushing end of said extendable portion and said stationary portion.

19. The system according to claim 18, wherein said sensor is at least one of: a strain gauge, a linear encoder, a pressure sensor, a proximity sensor, and an optical sensor.

20. The system according to claim 18, wherein said extendable portion comprises an extension mechanism and said extension mechanism is controllable via said computer.
